(19) United States
(12) Patent Application Publication
(10) Pub. No.: US 2024/0238411 A1
(43) Pub. Date: Jul. 18, 2024

(54) ADJUVANTED INACTIVATED RECOMBINANT RABIES VIRUS VECTORED CORONAVIRUS VACCINE FORMULATIONS

(71) Applicant: BHARAT BIOTECH INTERNATIONAL LIMITED, Hyderabad (IN)

(72) Inventors: Krishna Mohan VADREVU, Hyderabad (IN); Krishna Murthy ELLA, Hyderabad (IN); Ganneru BRUNDA, Hyderabad (IN); Srinivas VELLIMEDU KANNAPPA, Hyderabad (IN); Narasimha MULUGU, Hyderabad (IN)

(73) Assignee: BHARAT BIOTECH INTERNATIONAL LIMITED, Hyderabad (IN)

(21) Appl. No.: 18/565,210

(22) PCT Filed: May 31, 2022
(86) PCT No.: PCT/IN2022/050504

§ 371 Date: Nov. 29, 2023

(30) Foreign Application Priority Data
May 31, 2021 (IN) 202141021918

(51) Int. Cl. A61K 39/215; A61K 39/00; A61K 39/39; A61K 47/26; A61K 47/42; A61P 37/04

(52) U.S. Cl. CPC A61K 39/215; A61K 39/39; A61K 47/26; A61K 47/42; A61P 37/04; A61K 2039/5256; A61K 2039/55516; A61K 2039/55566

(57) ABSTRACT

The invention discloses an adjuvanted inactivated recombinant rabies virus vectored coronavirus vaccine formulation comprising SEPIVAC SWE or MemVax as adjuvant/s. The invention provides vaccine compositions, formulation 1 comprising combination of inactivated recombinant rabies virus vectored antigen and SEPIVAC SWE as an adjuvant and formulation 2 comprising combination of inactivated recombinant rabies virus vectored antigen and MemVax as an adjuvant. The said adjuvanted inactivated recombinant rabies virus vectored (rDNA-CoroRab) vaccine formulation prepared using SEPIVAC SWE or MemVax induces robust humoral, and cell mediated responses against SARS-CoV-2 compared to antigen alone and provides long term immunity.

### RELATED PATENT APPLICATION(S)

This application claims the priority to and benefit of Indian Patent Application No. 202141021918 filed on May 31, 2021; the disclosures of which are incorporated herein by reference.

### FIELD OF THE INVENTION

The invention relates to the field of vaccinology. Particularly the present invention relates to vaccine formulation/composition for COVID-19. More particularly, the present invention relates to vaccine composition comprising inactivated recombinant rabies virus expressing S1 domain of SARS-CoV-2 with one or more adjuvants or in combination (SEPIVAC SWE and/or MemVax). More particularly, the invention relates to the selection of adjuvants, method of preparation of vaccine composition and route of administration. More specifically, the invention describes the use of vaccine composition for preventing SARS-CoV-2 infections.

### BACKGROUND OF THE INVENTION

Currently the SARS-CoV-2 pandemic presents an extraordinary challenge to global health and hence it is of utmost importance to develop COVID-19 vaccine. More than 198 vaccines against SARS-CoV-2 are in preclinical trials, and over 156 candidate vaccines are in human trials as on 14 May 2022. They include inactivated, protein-, DNA-, mRNA-based vaccines and adenovirus and other viral vector-based vaccines.

Though, every approach has its own advantages and disadvantages, viral vector-based vaccines known for its excellent safety and the advantage of being typically cheaper to produce. However, viral vector-based vaccines are not always as immunogenic as replication-competent or replication-deficient viral vector vaccines and often require an adjuvant to increase their immunogenicity to an adequate level. Especially, in the context of COVID-19 vaccine, it is reported that an ideal COVID-19 vaccine composition should be able to induce Th1 biased immune response that can confer protection against SARS-CoV-2 infections. In such cases, selection of adjuvants is one of the biggest challenges in vaccine development. Moreover, formulation design and understanding the mechanism of action of an adjuvant against distinct types of pathogens and ensuring the desired type of immune response elicited by vaccine is also critical. Mainly, while optimizing the formulation, optimal concentrations of antigen and adjuvant that is sufficient to induce Th1 mediated response is utmost important. Of course, evaluation of efficacy and safety of vaccine composition are integral part of vaccine development. In this context, several adjuvants have been used as tailored made compositions, in order to achieve right vaccine composition, so as to improve the vaccine efficacy. Especially, during the pandemic scenario, where the COVID-19 vaccine developmental work has to be conducted unprecedented rapid speed, availability of GMP (Good manufacturing Practices) batch lots and safety profile of adjuvants is critical.

In general, mineral compounds (e.g. Aluminium hydroxide or Aluminium phosphate), water-in-oil or oil-in-water emulsions, or co-stimulatory molecules or immunostimulatory molecules or pathogen associated molecular patterns (PAMPs) that directly activate immune cells through specific receptors e.g. toll-like receptors (TLRs) have been used as an adjuvant, either as vaccine-delivery systems or immunomodulators. Immune cell activation followed by inflammatory responses amplify innate immunity and subsequently the activation of adaptive immune responses which facilitates cell mediated responses, so that vaccine will be more efficacious and long lasting. Emulsions as delivery systems are already used in life-saving vaccines as promising for various prophylactic vaccine applications, including seasonal and pandemic influenza vaccines.

In view of the above, an inactivated recombinant rabies virus (RABV) that express the S1 domain of the SARS-CoV-2 spike (S) protein (WO/2017/176596) used as antigen to prepare vaccine formulation. Recombinant rabies virus (RABV), wherein S1 domain of the SARS-CoV-2 is fused to part of the N terminal domain of the RABV glycoprotein (G). The RABV vaccine itself has been safely used for decades in more than 100 million people, including children, pregnant women, and the elderly, and proven to result in long-lasting immunity.

While knowing the limitation of this viral vector vaccine platform, present invention discloses the use of adjuvants in the preparation of an adjuvanted inactivated rabies virus vectored coronavirus vaccine formulation.

SEPIVAC SWE is an Oil in water emulsion, which is free of Intellectual Property Rights. This adjuvant has been developed by the Vaccine Formulation Laboratory for technology transfer in order to empower developing countries vaccine manufacturers. Over the years, SWE has demonstrated safe and effective, both in terms of enabling adjuvanticity and dose-sparing, when included in numerous preclinical vaccine candidates, including against Inactivated Polio Virus, Influenza, Respiratory Syncytial Virus, Rabies, and Group A streptococcus. Similar proprietary Oil-in-water emulsions adjuvants such as AS03 (GlaxoSmithKline) and MF59 (Novartis) have been shown to improve the immune response in Humans However, low-income countries face the challenge of lack of access to proprietary adjuvants.

MemVax (Ad-ISF35) is a replication incompetent adenovirus type 5 (Ad 5), E1 deleted/partial E3 deleted, vector encoding human/mouse chimeric CD40 ligand membrane-stable chimeric CD154 (University of Wisconsin-Madison, USA). Ad-ISF35 has shown to promote immune recognition and anti-leukemia T-cell activation. leading to tumor regression, same has not been tried before for the Vaccine development (Cancer gene therapy, 19(5), 336-344).

WO2017079297A1 teaches that administration of the expression vector encoding CD154 enhances the expression of costimulatory and death receptor molecules PD-L1 and/or PD-L2 on the cancer cells, making the cells susceptible to treatment with inhibitors of the PD-1 pathway. Further the WO2018191369A1 discloses administering to the individual an effective amount of a TLR agonist and a chimeric CD154 polypeptide for enhanced immune functions for treating cancer. Though the adjuvant MemVax has been used for treatment of cancer, same has not been tried before for the Vaccine development.

With this background, present invention deals with the use of two cost effective adjuvants (SEPIVAC SWE and MemVax), in developing the Adjuvanted Corona Virus vaccine (rDNA-CoroRab), against COVID-19, to induce cell mediated responses

### OBJECTS OF THE INVENTION

The primary object of the present invention is to provide an adjuvanted inactivated rabies virus vectored coronavirus vaccine formulation, particularly an adjuvanted inactivated rabies virus vectored (rDNA-CoroRab) vaccine against COVID-19.

Another object of the invention is to develop and prepare an adjuvanted Coronavirus Vaccine formulation using SEPIVAC SWE or MemVax as adjuvant/s.

Another object of the invention is to provide and use of various stabilizers in the said formulation.

Another object of the invention is to provide and use of an adjuvanted Coronavirus Vaccine formulation to enhance the efficacy of the vaccine, while increasing both humoral and cell mediated immune responses or Th1/Th2 immune response.

Another object of the invention is to assess ability of the said formulation to induce cell mediated T cell responses and IgA titers.

Another object of the invention is to provide long term immunity using SEPIVAC SWE or MemVax as adjuvant/s in the preparation of Adjuvanted Coronavirus Vaccine (rDNA-CoroRab).

Yet another object of the invention is to provide a method of preparation of coronavirus vaccine formulations using two adjuvants, SEPIVAC SWE and/or MemVax, with inactivated rabies virus vector, which express S1 domain of SARS-CoV-2 to increase the effectiveness of the vaccine.

A further object of the present invention is to provide an adjuvanted inactivated recombinant rabies virus vectored (rDNA-CoroRab) vaccine formulation prepared using SEPIVAC SWE and/or MemVax, induces robust humoral and cell mediated responses against SARS-CoV-2 compared to antigen alone and provides long term immunity.

Yet another object of the invention is to provide a method of treatment against coronavirus disease and/or COVID-19 by using and administering above said adjuvanted inactivated rabies virus vectored coronavirus vaccine formulation of present invention, particularly by using and administering an adjuvanted inactivated recombinant rabies virus vectored (rDNA-CoroRab) vaccine such as vaccine formulation 1 and/or formulation 2 of present invention against COVID-19.

Yet further object of the present invention is to provide the adjuvanted Coronavirus vaccine Formulations effective against SARS-CoV-2 variants or variants of Concern (VOCs) such as alpha, Beta, Delta, and omicron.

### SUMMARY OF THE INVENTION

The present invention relates to development of an adjuvanted inactivated rabies virus vectored coronavirus vaccine formulation.

Accordingly, the present invention describes and discloses the use of SEPIVAC SWE or MemVax as adjuvants in the preparation of adjuvanted inactivated rabies virus vectored (rDNA-CoroRab) vaccine against COVID-19 and its functional characterization.

In one aspect present invention provides an adjuvanted vaccine formulation against COVID-19 infection, comprising:
a. vaccine antigen, wherein the antigen is an inactivated rabies virus vector expressing S1 domain of SARS-CoV-2 Spike Protein;
b. one or more adjuvants selected from SEPIVAC SWE and MemVax;
c. optionally, one or more stabilizers; and
d. optionally, one or more preservatives.

In the said formulation, SEPIVAC SWE is an oil in water emulsion adjuvant; and MemVax is a replication incompetent adenovirus type 5 (Ad 5), E1 deleted/partial E3 deleted, vector encoding human/mouse chimeric CD40 ligand.

In the said adjuvanted vaccine formulation, one or more stabilizers are selected from Trehalose, Sucrose, Maltose, Human Serum Albumin (HSA) or combination thereof.

The concentration of Trehalose is between 0.5%-5% (w/v); Maltose is between 5% to 10% (w/v); Sucrose is between 0-70% (w/v) and HSA is between 0.5%-5% (w/v).

In the said adjuvanted vaccine formulation one or more preservatives are selected from:
0.1% to 0.5% (w/v) 2-phenoxyethanol;
0.003% to 0.01% (w/v) thiomersal;
0.01% to 0.1% (w/v) methyl p-hydroxybenzoate;
0.01 to 0.1% (w/v) propyl p-hydroxybenzoate; and
combinations thereof.

In the said adjuvanted vaccine formulation antigen and SEPIVAC SWE or MemVax are present in 1:1 ratio (v/v). The antigen concentration in the said formulation ranges from 5 to 50 μg.

SEPIVAC SWE in the present formulation comprises of squalene in the concentration ranging from 1 to 5% (v/v), preferably 2.5 to 4.5% (v/v).

MemVax in the present formulation comprises of recombinant adenovirus type 5 (Ad 5), vector encoding human/mouse chimeric CD40 ligand ranging from 0.5×10¹⁰ vp/dose to 5×10¹⁰ vp/dose (virus particles/dose), preferably 0.5×10¹⁰ vp/dose to 3×10¹⁰ vp/dose.

The said vaccine formulation induces robust humoral and cell mediated response against COVID-19 infection compared to antigen alone and provides long term immunity.

The said vaccine formulation elicits high humoral protective response compared to antigen alone in mammals.

The said vaccine formulation elicits high Cell meditated or TH1/TH2 immunity compared to antigen alone in mammals.

The said vaccine formulation induces TH1 mediated immune response, indicated by robust induction of IFN-γ and TNF-α in mammals.

In another aspect present invention discloses a method of preparing an adjuvanted vaccine formulation against COVID-19 infection, comprising:
a. providing an antigen, wherein the antigen is an inactivated rabies virus vector expressing S1 domain of SARS-CoV-2 Spike Protein; and
b. providing one or more adjuvants selected from SEPIVAC SWE and MemVax;
c. mixing the antigen and adjuvant in 1:1 ratio (v/v);
d. optionally, mixing one or more stabilizers; and
e. optionally, mixing one of more preservatives.

In the said method, SEPIVAC SWE is an oil in water emulsion adjuvant; and MemVax is a replication incompetent adenovirus type 5 (Ad 5), E1 deleted/partial E3 deleted, vector encoding human/mouse chimeric CD40 ligand.

In the said method, the antigen concentration ranges from 5 to 50 μg.

SEPIVAC SWE in the present method comprises of squalene in the concentration ranging from 1 to 5% (v/v), preferably 2.5 to 4.5% (v/v).

MemVax in the present method comprises of recombinant adenovirus type 5 (Ad 5), vector encoding human/mouse chimeric CD40 ligand ranging from 0.5×10¹⁰ vp/dose to 5×10¹⁰ vp/dose (virus particles/dose), preferably 0.5×10¹⁰ vp/dose to 3×10¹⁰ vp/dose

In the said method, one or more stabilizers are selected from Trehalose, Sucrose, Maltose, Human Serum Albumin (HSA) or combination thereof.

The concentration of Trehalose is between 0.5%-5% (w/v); Maltose is between 5% to 10% (w/v); Sucrose is between 0-70% (w/v) and HSA is between 0.5%-5% (w/v).

In the said method, one or more preservatives are selected from:
0.1% to 0.5% (w/v) 2-phenoxyethanol;
0.003% to 0.01% (w/v) thiomersal;
0.01% to 0.1% (w/v) methyl p-hydroxybenzoate;
0.01 to 0.1% (w/v) propyl p-hydroxybenzoate; and
combinations thereof.

Yet in another aspect present invention discloses the use of adjuvants SEPIVAC SWE and/or MemVax in developing adjuvanted COVID-19 vaccine, in which gene of S1 fragment of Spike Protein of SARS-CoV-2 has been inserted between N and P genes of attenuated rabies virus.

Yet in further aspect present invention discloses an adjuvanted vaccine formulation effective against SARS-CoV-2 variants or variants of Concern (VOCs) such as alpha, Beta, Delta, and omicron.

### BRIEF DESCRIPTION OF FIGURES

### Formulation 1:

### Humoral Immune Response:

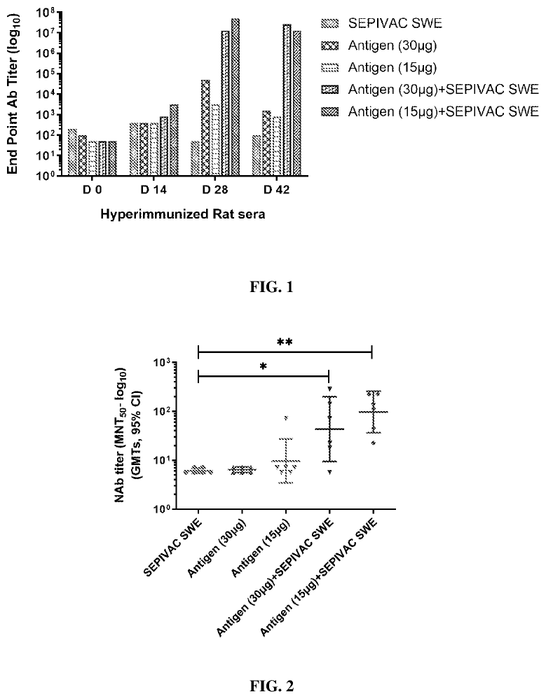

FIG. 1: Spike Specific Antibody titers elicited by the Formulation 1.

FIG. 2: Neutralization antibody titers elicited by Formulation 1.

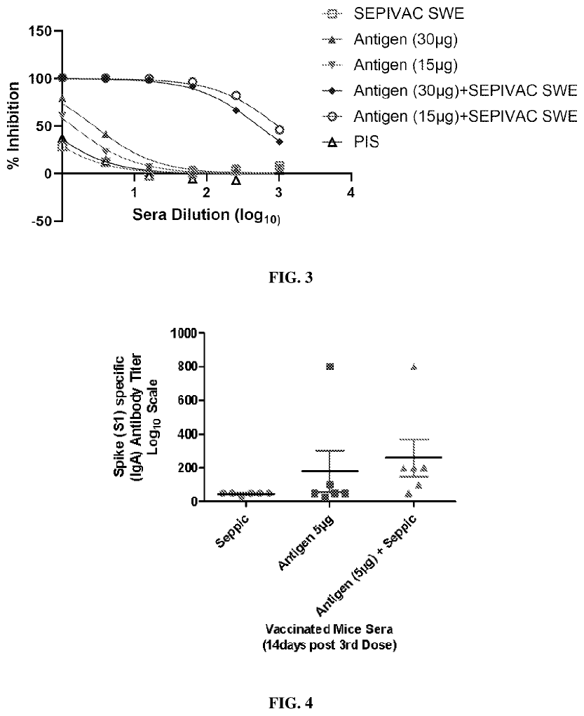

FIG. 3: Surrogate Virus neutralization Titer (sVNT) by Formulation 1.

FIG. 4: IgA titers elicited against Adjuvanted Coronavirus vaccine by Formulation 1.

### Formulation 2:

### Humoral Immune Response:

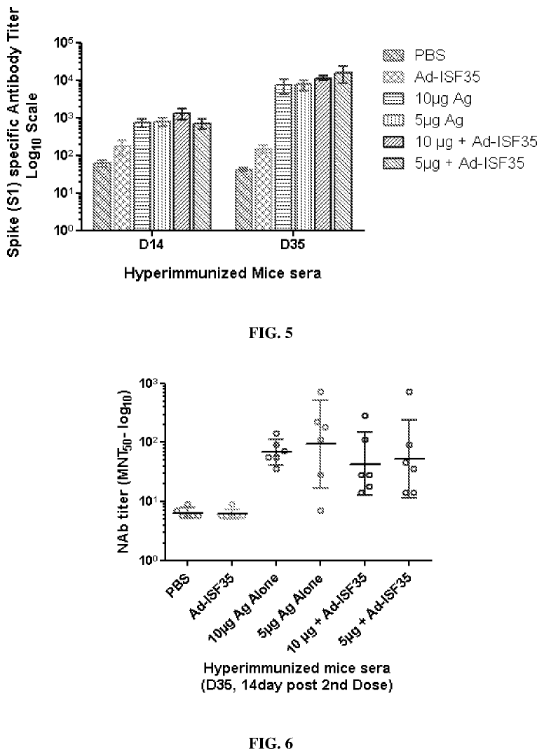

FIG. 5: Spike Specific Antibody titers elicited by the Formulation 2.

FIG. 6: Neutralization antibody titers elicited by Formulation 2.

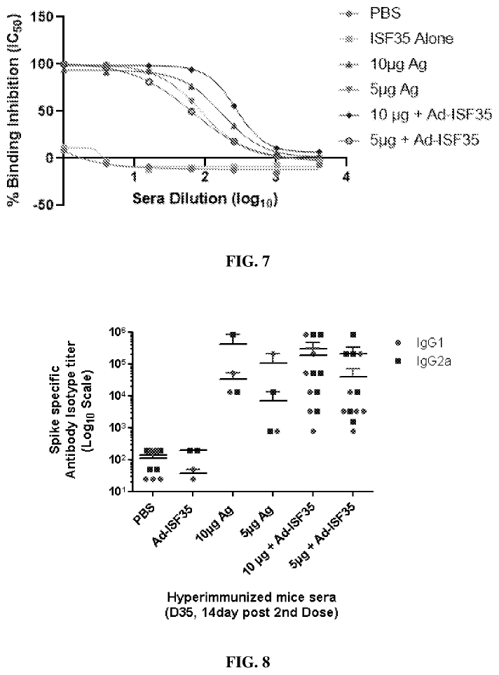

FIG. 7: Surrogate Virus neutralization Titer (sVNT) by Formulation 2.

### Cell Mediated Immune Response:

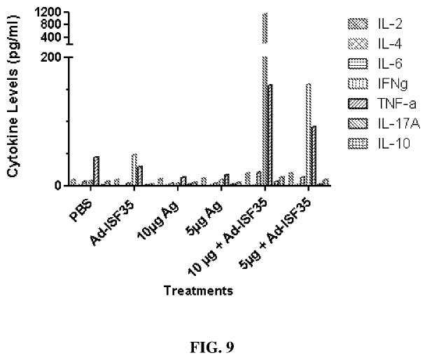

FIG. 8: Immunoglobulin Subclasses induced by Formulation 2 (Ab Isotyping).

FIG. 9: Cytokine Profile induced by Formulation 2.

### DETAILED DESCRIPTION OF THE INVENTION

It is to be understood that the disclosed vaccine formulation or compositions in the art may be tailor made with various adjuvants at different concentrations and antigen at various concentrations for the applications described in the present invention.

The invention describes generation of an adjuvanted inactivated recombinant rabies virus vectored (rDNA-CoroRab) vaccine formulation against COVID-19.

In one aspect, the present invention provides an adjuvanted inactivated recombinant rabies virus vectored coronavirus vaccine formulation comprising SEPIVAC SWE or MemVax as adjuvant/s.

In yet another aspect, the present invention provides formulation 1 comprising combination of inactivated recombinant rabies virus vectored antigen, namely coronavirus vaccine (rDNA-BBV151) and SEPIVAC SWE as an adjuvant.

In yet another aspect, the present invention provides formulation 2 comprising combination of inactivated recombinant rabies virus vectored antigen, namely coronavirus vaccine (rDNA-BBV151) and MemVax as an adjuvant.

In yet another aspect, the present invention provides a method of preparation of formulation using two adjuvants, SEPIVAC SWE and MemVax, with inactivated recombinant rabies virus vector, which express spike (S1) of SARS-CoV-2 to increase the effectiveness of the vaccine.

The said formulation comprises combination of inactivated recombinant rabies virus vectored antigen, namely Coronavirus vaccine (rDNA-BBV151) and adjuvant, wherein the antigen is a BBV151-inactivated recombinant rabies virus vector expressing spike protein.

Specifically, the invention describes an adjuvanted Coronavirus Vaccine formulation using SEPIVAC SWE or MemVax as adjuvant/s.

In one embodiment the present invention describes the use of SEPIVAC SWE, and/or MemVax as adjuvant/s in the preparation of formulation.

In one embodiment the present invention also describes the ability of each adjuvant to induce both humoral and cell mediated responses.

Use of two adjuvants in developing the Adjuvanted Coronavirus Vaccine (rDNA CoroRab), against SARS-CoV-2, in which gene of S1 fragment of spike protein of SARS-CoV-2 has been inserted between N and P genes of attenuated rabies virus (SAD B19). In the present invention, said two different adjuvants are used to induce cell mediated responses or to increase the humoral antibody response.

Further the said two adjuvants used in the present invention are as below:
SEPIVAC SWE: SEPIVAC SWE is an oil in water emulsion purchased from SEPPIC, France. It comprises of 4.1% (v/v) Squalene and surfactants. SEPIVAC SWE is an Oil in water emulsion purchased from SEPPIC, France.
MemVax (Ad-ISF35): MemVax (Ad-ISF35) is a replication incompetent adenovirus type 5 (Ad 5), E1 deleted/partial E3 deleted E1 deleted/partial E3 deleted, vector encoding human/mouse chimeric CD40 ligand. This cDNA transgene was expressed by the human Cytomegalovirus promotor/enhancer. Bharat Biotech International Limited obtained the adjuvant “MemVax” under Material Transfer Agreement with Memgen Inc., Houston, Texas, USA.

These adjuvants are used in developing the Adjuvanted Coronavirus Vaccine (rDNA-CoroRab), against SARS-CoV-2, to induce cell mediated responses.

The present invention further describes the use of SEPIVAC SWE and MemVax as adjuvants in the preparation of formulation. The present invention also describes the ability of each adjuvant to induce both humoral and cell mediated responses.

### Vaccine Formulations:

According to the present invention two adjuvants SEPIVAC SWE and MemVax, have been used in developing the Adjuvanted Coronavirus Vaccine (rDNA-CoroRab), against SARS-CoV-2, in which gene of S1 fragment of spike protein of SARS-CoV-2 has been inserted between N and P genes of attenuated rabies virus (SAD B19). The above said two different adjuvants are used to induce cell mediated responses or to increase the humoral antibody response.

Accordingly, present invention discloses an adjuvanted vaccine formulation against COVID-19 infection, comprising:
a. vaccine antigen, wherein the antigen is an inactivated rabies virus vector expressing S1 domain of SARS-CoV-2 Spike Protein;
b. one or more adjuvants selected from SEPIVAC SWE and MemVax;
c. optionally, one or more stabilizers; and
d. optionally, one or more preservatives.

In the said formulation, SEPIVAC SWE is an oil in water emulsion adjuvant; and MemVax is a replication incompetent adenovirus type 5 (Ad 5), E1 deleted/partial E3 deleted, vector encoding human/mouse chimeric CD40 ligand.

The said vaccine composition may or may not contain stabilizers. The stabilizers may include the one or two or three or combination of all stabilizers. The stabilizers may be selected from Trehalose, Sucrose, Maltose, Human Serum Albumin (HSA) or combination thereof. The concentration of Trehalose and HSA may range from 0.5 to 5% (w/v), whereas concentration of Maltose is between 5-10% (w/v) and sucrose may range up to 70% (w/v).

The said vaccine formulation may or may not contain preservatives. The preservatives include 2-phenoxyethanol (1 to 5 mg/ml) or thiomersal (0.003 to 0.01% (w/v)) or methyl p-hydroxybenzoate (0.01 to 0.1% (w/v)) or propyl p-hydroxybenzoate (0.01 to 0.1% (w/v)) or combinations thereof.

In the said vaccine formulation, the antigen and SEPIVAC SWE and/or MemVax are present in 1:1 ratio (v/v). The antigen concentration in the present vaccine formulation may range from 5 to 50 μg.

The vaccine formulation of the present invention induces robust humoral and cell mediated response against COVID-19 infection compared to antigen alone and provides long term immunity.

The vaccine formulation of the present invention elicits high humoral protective response and high Cell meditated or TH1/TH2 immunity compared to antigen alone in mammals.

The said vaccine formulation induces TH1 mediated immune response, indicated by robust induction of IFN-γ and TNF-α in mammals.

The vaccine formulation of the present invention is effective against SARS-CoV-2 variants or variants of Concern (VOCs) such as alpha, Beta, Delta, and omicron.

### Method of Preparation of Formulation:

The present invention further discloses the method of preparation of formulation using two adjuvants, SEPIVAC SWE and MemVax (Ad-ISF35), with inactivated rabies virus vector, which express spike (S1) of SARS-CoV-2 to increase the effectiveness of the vaccine.

Accordingly, present invention discloses a method of preparing an adjuvanted vaccine formulation against COVID-19 infection, comprising:
a. providing an antigen, wherein the antigen is an inactivated rabies virus vector expressing S1 domain of SARS-CoV-2 Spike Protein; and
b. providing one or more adjuvants selected from SEPIVAC SWE and MemVax;
c. mixing the antigen and adjuvant in 1:1 ratio (v/v);
d. optionally, mixing one or more stabilizers; and
e. optionally, mixing one of more preservatives.

In the said method, SEPIVAC SWE is an oil in water emulsion adjuvant; and MemVax is a replication incompetent adenovirus type 5 (Ad 5), E1 deleted/partial E3 deleted, vector encoding human/mouse chimeric CD40 ligand.

The antigen concentration may range from 5 to 50 μg.

The method of the present invention comprises mixing of Inactivated recombinant Rabies virus vectored antigen with either of the adjuvants in ratio 1:1 (v/v), wherein the said antigen is a BBV151-inactivated recombinant rabies virus vector expressing spike protein, forming an antigen solution.

The said antigen solution may or may not contain stabilizers. The stabilizers may include the one or two or three or combination of all stabilizers. The stabilizers may be selected from Trehalose, Sucrose, Maltose, Human Serum Albumin (HSA) or combination thereof. The concentration of Trehalose is between 0.5%-5% (w/v); Maltose is between 5% to 10% (w/v); Sucrose is between 0-70% (w/v) and HSA is between 0.5%-5% (w/v).

The said antigen solution may or may not contain preservatives. The preservatives include 2-phenoxyethanol (1 to 5 mg/ml) or thiomersal (0.003 to 0.01% (w/v)) or methyl p-hydroxybenzoate (0.01 to 0.1% (w/v)) or propyl p-hydroxybenzoate (0.01 to 0.1% (w/v)) or combinations thereof.

In one embodiment, the formulation 1 contains inactivated recombinant rabies virus vector, which express spike (S1) of SARS-CoV-2 and SEPIVAC SWE as an adjuvant.

In one embodiment, the formulation 2 contains inactivated recombinant rabies virus vector, which express spike (S1) of SARS-CoV-2 and MemVax (Ad-ISF35) as an adjuvant.

In one embodiment the Formulations 1 and 2 may or may not contain stabilizers.

In one embodiment the Formulations 1 and 2 may or may not contain preservatives.

In one embodiment the Formulation 1 and 2 may contain 0.003% to 0.01% (w/v) thimerosal.

In one embodiment the Formulation 1 and 2 may contain 0.1% to 0.5% (w/v) 2-Phenoxyethanol.

In one embodiment the Formulation 1 and 2 may contain max 0.02% to 0.1% (w/v) methyl 4-hydroxy benzoate.

In one embodiment the Formulation 1 and 2 may contain max 0.02% to 0.1% (w/v) propyl 4-hydroxy benzoate.

### Adjuvants:

### SEPIVAC SWE:

In one embodiment SEPIVAC SWE is used as an adjuvant along with the Inactivated recombinant rabies virus vectored vaccine. The present invention discloses the use of SEPIVAC SWE as an adjuvant.

Present invention discloses the preparation of formulation 1 comprising combination of inactivated recombinant rabies virus vectored antigen, namely coronavirus vaccine (rDNA-BBV151) and SEPIVAC SWE as an adjuvant. The method comprises of mixing of the Antigen and adjuvant (SEPIVAC SWE) in suitable ratio.

In the said formulation 1, the SEPIVAC SWE comprises of squalene in the concentration which ranges from 1 to 5% (v/v), preferably 2.5 to 4.5% (v/v).

In one embodiment, the present invention further discloses physico-chemical characteristics of antigen and adjuvant in the said formulation 1 and describes the humoral and cell mediated responses elicited by the formulation 1 made with SEPIVAC SWE.

In one embodiment, the stability and safety of the formulation 1 i.e. adjuvanted coronavirus Vaccine made with SEPIVAC SWE has been evaluated in the present invention.

### MemVax (Ad-ISF35):

In another embodiment MemVax (Ad-ISF35) is used as an adjuvant along with the Inactivated recombinant rabies virus vectored vaccine.

Present invention discloses the preparation of formulation 2 comprising combination of inactivated recombinant rabies virus vectored antigen, namely coronavirus vaccine (rDNA-BBV151) and MemVax (Ad-ISF35) as an adjuvant. The method comprises of mixing of the Antigen and adjuvant (Ad-ISF35) in suitable ratio.

In the said formulation 2, MemVax comprises of recombinant adenovirus type 5 (Ad 5) vector encoding human/mouse chimeric CD40 ligand in the concentration which ranges from 0.5×10¹⁰ vp/dose to 5×10¹⁰ vp/dose, preferably 0.5×10¹⁰ vp/dose to 3×10¹⁰ vp/dose.

In one embodiment, the present invention further discloses physico-chemical characteristics of antigen and adjuvant in the formulation 2 and describes the humoral and cell mediated responses elicited by the formulation 2 made with MemVax (Ad-ISF35).

Further in one embodiment the stability and safety of the formulation 2 i.e. adjuvanted coronavirus Vaccine made with MemVax (Ad-ISF35) has been evaluated.

### Advantages

Advantages of the said adjuvanted inactivated recombinant rabies virus vectored coronavirus vaccine formulations:
Adjuvanted vaccine Formulations (1 and 2) prepared using either with SEPIVAC SWE (Formulation 1) or MemVax (Ad-ISF35, Formulation 2) induces robust humoral and cell mediated responses against SARS-CoV-2 compared to antigen alone.
Adjuvanted Coronavirus Vaccine Formulations (1 and 2) also induces moderate levels of IgA in serum, when tested in vaccinated mice.
Adjuvanted Coronavirus vaccine Formulations (1 and 2) would help in reducing the antigen concentration required, thereby reduces the cost.
Adjuvanted Coronavirus vaccine Formulations with SEPIVAC SWE found to help in slow and sustained release of antigen to antigen presenting cells.
Adjuvanted Coronavirus vaccine Formulations (1 and 2) provides long term immunity.
Adjuvanted Coronavirus vaccine Formulations (1 and 2) effective against SARS-CoV-2 variants or variants of Concern (VOCs) such as alpha, Beta, Delta, and omicron.

### EXAMPLES

### Examples for Formulation 1

FORMULATION 1: Combination of Inactivated recombinant Rabies virus vectored antigen, namely Coronavirus vaccine (rDNA-BBV151) and SEPIVAC SWE as an adjuvant. SEPIVAC SWE is an Oil in water emulsion, purchased from SEPPIC, France.

### Example 1: Preparation of Formulation 1 Using SEPIVAC SWE as Adjuvant

Adjuvanted Coronavirus Vaccine formulation 1 was prepared by mixing the Antigen and adjuvant (SEPIVAC SWE) in a ratio 1:1 (v/v). Antigen concentration in the above said formulation 1 may range from 5 to 50 μg and adjuvant (SEPIVAC SWE) comprise different concentration of squalene 1 to 5% (v/v), preferably 2.5 to 4.5% (v/v).

Antigen, which is a BBV151-inactivated recombinant rabies virus vector expressing spike protein solution may or may not contain stabilizers. The stabilizers may include the one or two or three or combination of all stabilizers. The stabilizers may be selected from Trehalose, Sucrose, Maltose, Human Serum Albumin (HSA) or combination thereof. The concentration of Trehalose and HSA may range from 0.5 to 5% (w/v), whereas concentration of Maltose is between 5-10% (w/v); and sucrose may range up to 70% (w/v).

This antigen solution may or may not contain preservatives. The preservatives include 2-phenoxyethanol (1 to 5 mg/ml) or thiomersal (0.003 to 0.01% (w/v)) or methyl p-hydroxybenzoate (0.01 to 0.1% (w/v)) or propyl p-hydroxybenzoate (0.01 to 0.1% (w/v)) and their combinations thereof.

### Example 1.1: Physico-Chemical Characterization of Formulation 1

Formulation was characterized for quantification of Total protein and spike (S1), S1 expression, particle size, etc. by Lowry method, ELISA, western blot and zeta sizer etc., and the formulation 1 found stable.

### Example 1.2: Animals

Wistar Rats (6-8 week old, Equal Gender) were purchased and maintained in the animal care facility under standard approved protocols. All procedures involving mice were carried out with the approval of Institutional Animal Ethics Committee.

### Example 1.2.1: Immunization

In this example, rats were vaccinated to evaluate the immunogenicity of an adjuvanted vaccine formulations (at 30 μg and 15 μg antigen concentration). For this, rats were administered intramuscularly with full HSD (full human intended single dose) of adjuvanted coronavirus vaccine formulations containing 30 μg and 15 μg antigen concentration/0.4 ml/rat) on day 0, 14, 28 and 42. Blood was collected on various time points, either before the immunization (Day 0) or 14 Days post immunization (Day 14, 28 and 42). Sera was separated and stored at −20° C. until further use.

### Example 1.2.2: Humoral Response

Enzyme Linked Immunosorbent Assay (ELISA): Sera collected at different time points were pooled group wise and used to determine spike specific antibody titers by ELISA. Antigen alone elicited less or negligible antibody titers, whereas adjuvanted Coronavirus vaccine formulation, (i.e., BBV151 with SEPIVAC SWE adjuvant, namely Formulation 1) elicited high spike (S1) specific antibody titers (shown in FIG. 1).

Micro-neutralization Antibody Titers (MNT₅₀): Individual sera collected from all groups on Day 28, was used to test neutralization antibody titers by MNT₅₀. These results indicated that the antigen alone elicited less or negligible neutralization antibody (NAb) titers, whereas adjuvanted Coronavirus vaccine formulation i.e. BBV151 with SEPIVAC SWE adjuvant, (namely Formulation 1) elicited high NAb titers compared to antigen alone at two concentrations (30 and 15 μg) and these titers are significantly high compared to placebo (SEPIVAC SWE alone) (shown in FIG. 2). Statistical analysis was performed using Mann-whitney two tailed test at a p value<0.05 (*) and <0.001(**) respectively.

SARS-CoV-2 Surrogate Virus Neutralization Test (sVNT): Surrogate Assay was performed using cPass™ SARS-CoV-2 Neutralization Antibody Detection Kit (GenScript), which is designed to detect SARS-CoV-2 neutralizing antibodies. To perform this assay, pooled sera of Day 28 from all group animals was used and determined the % Inhibition at 50%, (i.e. ability of antibodies present in the hyperimmunized sera to inhibit the binding RBD to ACE2 protein). The dose response inhibition curve was generated using non-linear regression analysis (shown in FIG. 3). Adjuvanted Coronavirus vaccine showed high % IC₅₀ values compared to antigen alone (shown in Table 1)

TABLE 1

| Group | IC₅₀ |
|---|---|
| SEPIVAC SWE | 0.408 |
| Antigen (30 μg) | 2.771 |
| Antigen (15 μg) | 1.385 |
| Antigen (30 μg) + SEPIVAC SWE | 527 |
| Antigen (15 μg) + SEPIVAC SWE | 972.1 |
| Pre-Immune Sera | 0.565 |

### Example 1.2.3: Cell Mediated Response

Immunoglobulin Subclass (Antibody Isotyping) Analysis: Pooled sera (Day 28) collected from vaccinated rats were used to perform ELISA to analyze spike (S1) specific IgG1 and IgG2b antibody titers and End point titer of these Immunoglobulin subclasses was used to calculate Th1:Th2 Index using the formula IgG2b/IgG1. These results indicated that immune response is Th1 biased, based on the Th1: Th2 index≥1 (shown in Table 2). Hence, it was concluded that adjuvanted Coronavirus vaccine formulation elicits both humoral and cell mediated responses, though further investigations need to be evaluated. Moderate levels of IgA titers were also found in serum, which were determined by ELISA (FIG. 4).

TABLE 2

| | Th1 | Th2 | Th1:Th2 |
|---|---|---|---|
| Description | IgG2b | IgG1 | Index |
| SEPIVAC SWE alone | 50 | 200 | 0.25 |
| Antigen Alone (30 μg) | 25600 | 800 | 32.00 |
| Antigen Alone (15 μg) | 1600 | 200 | 8.00 |
| Antigen (30 μg) + SEPIVAC SWE | 204800 | 51200 | 4.00 |
| Antigen (15 μg) + SEPIVAC SWE | 204800 | 204800 | 1.00 |

### Example 1.2.4: Safety Evaluation

To establish safety of adjuvanted Coronavirus Vaccine formulation 1, repeated dose 5 toxicity test was performed in Rats and Rabbits. Parameters such as clinical signs, body weight, feed consumption, body temperature, clinical pathology, terminal body weights, organ weights and gross pathology were observed during the entire experimental period and there were no treatment related changes were found. Further, there were no treatment related microscopic findings observed during the histopathological investigations.

In conclusion, adjuvanted Coronavirus vaccine with SEPIVAC SWE as an adjuvant forming a formulation was found to be immunogenic and elicits both humoral and cell mediated immune responses with a good neutralization antibody titers.

### Examples for Formulation-2

FORMULATION 2: Combination of Inactivated recombinant Rabies virus vectored antigen, namely Coronavirus vaccine (rDNA-BBV151) and Ad-ISF35 as an adjuvant. MemVax (AdISF35) is a replication incompetent adenovirus type 5 (Ad 5), E1 deleted/partial E3 deleted, vector encoding human/mouse chimeric CD40 ligand. This cDNA transgene was expressed by the human Cytomegalovirus promotor/enhancer. Bharat Biotech International Limited obtained the adjuvant “MemVax” under Material Transfer Agreement with Memgen Inc, Houston, Texas, USA.

### Example 2: Preparation of Formulation 2 Using MemVax (Ad-ISF35) as Adjuvant

Adjuvanted Coronavirus Vaccine formulation 2 was prepared by mixing the Antigen and adjuvant (Ad-ISF35) in a ratio 1:1 (v/v), wherein concentration of recombinant adenovirus type 5 (Ad 5), vector encoding human/mouse chimeric CD40 ligand in MemVax may range from 0.5×10¹⁰ vp/dose to 5×10¹⁰ vp/dose, preferably 0.5×10¹⁰ vp/dose to 3×10¹⁰ vp/dose and antigen concentration in the above said formulation 2 may range from 5 to 50 μg.

Antigen, which is a BBV151-inactivated recombinant rabies virus vector expressing spike protein solution may or may not contain stabilizers. The stabilizers may include the one or two or three or combination of all stabilizers. The stabilizers may be selected from Trehalose, Sucrose, Maltose, Human Serum Albumin (HSA) or combination thereof. The concentration of Trehalose is between 0.5%-5% (w/v); Maltose is between 5% to 10% (w/v); Sucrose is between 0-70% (w/v); and HSA is between 0.5%-5% (w/v).

This antigen solution may or may not contain preservatives. The preservatives include 2-phenoxyethanol (1 to 5 mg/ml) or thiomersal (0.003 to 0.01% (w/v)) or methyl p-hydroxybenzoate (0.01 to 0.1% (w/v)) or propyl p-hydroxybenzoate (0.01 to 0.1% (w/v)) and their combinations thereof.

### Example 2.1: Physio-Chemical Characterization of Formulation 2

Formulation was characterized for quantification of Total protein and spike (S1), S1 expression, particle size, etc., by Lowry method, ELISA, western blot and Zeta sizer etc., and the formulation 2 found stable.

### Example 2.2: Animals

BALB/c (6-8 week old, Equal Gender) mice were purchased and maintained in the animal care facility under standard approved protocols. All procedures involving rats were carried out with the approval of Institutional Animal Ethics Committee.

### Example 2.2.1: Immunization

In this example, BALB/c were vaccinated to evaluate the immunogenicity of an adjuvanted vaccine formulation 2 (at 10 μg and 5 μg antigen concentration). For this, BALB/c (n=6) mice were administered intramuscularly with ⅓^{rd} HSD (human intended single dose) of adjuvanted corona virus vaccine formulation 2 (containing 10 μg or 5 μg antigen along with Ad-ISF35 as an adjuvant/0.1 ml/mouse) on day 0 and 21. Blood was collected on various time points, either before the immunization (Day 0) or 14 Days post immunization (Day 14 and 35). Sera was separated and stored at −20° C. until further use.

### Example 2.2.2: Humoral Response

Enzyme Linked Immunosorbent Assay (ELISA): Individual sera collected at different time points were used to determine spike specific antibody titers by ELISA. Both Antigen and adjuvanted Coronavirus vaccine formulation 2, (i.e., BBV151 with Ad-ISF35 adjuvant, namely Formulation 2) elicited high spike (S1) specific antibody titers (shown in FIG. 5).

Micro-neutralization Antibody Titers (MNT₅₀): Individual sera collected from all groups on Day 35, was used to test neutralization antibody titers by MNT₅₀. These results indicated that both Antigen and adjuvanted Coronavirus vaccine formulation 2, (i.e., BBV151 with Ad-ISF35 adjuvant, namely Formulation 2) elicited high NAb titers (shown in FIG. 6).

SARS-CoV-2 Surrogate Virus Neutralization Test (sVNT): Surrogate Assay was performed using cPass™ SARS-CoV-2 Neutralization Antibody Detection Kit (GenScript), which is designed to detect SARS-CoV-2 neutralizing antibodies. To perform this assay, pooled sera of Day 28 from all group animals was used and determined the % Inhibition at 50%, (i.e. ability of antibodies present in the hyperimmunized sera to inhibit the binding RBD to ACE2 protein). The dose response inhibition curve was generated using non-linear regression analysis (shown in FIG. 7). Adjuvanted Coronavirus vaccine showed high % IC₅₀ values compared to antigen alone (shown in Table 3).

TABLE 3

| Group | IC₅₀ |
|---|---|
| PBS | 0.011 |
| Adjuvant (Ad-ISF35) | 3.574 |
| Antigen (10 μg) | 174.6 |
| Antigen (5 μg) | 88.54 |
| Antigen (10 μg) + Ad-ISF35 | 271.8 |
| Antigen (5 μg) + Ad-ISF35 | 65.80 |

### Example 2.2.3: Cell Mediated Response

Antibody Isotyping: Individual sera collected on Day 35 from animals of all groups were used to perform ELISA to analyze spike (S1) specific IgG1 and IgG2a antibody titers and End point titer of these Immunoglobulin subclasses was determined. These results showed that ≥50% animals showed IgG2a titer than IgG1, indicating Th1 biased response (shown in FIG. 8).

Cytokine Bead Array (CBA) Assay: To analyze type of immune response elicited by the adjuvanted Corona Virus vaccine, Splenocytes from vaccinated mice were stimulated with inactivated SARS-CoV-2 antigen or S1 protein, for 72 hrs. Culture supernatant was tested for Th1/Th2/Th17 cytokines using mouse CBA kit, BD Bioscience. Interestingly, it was found that adjuvanted corona virus vaccine formulation 2 found to induce Th1 mediated immune response, which was demonstrated by the robust induction of IFN-γ and TNF-α (shown in FIG. 9).

### Example 2.2.4: Safety Evaluation

Animals, both BALB/c mice and Wistar Rats were administered with two doses of Formulation 2 found safe with no mortality during the entire experimental period. No abnormal clinical signs, no abnormal body weight gain noticed. Feed consumption was normal.

In conclusion, adjuvanted Coronavirus vaccine with formulation 2 found to be immunogenic and elicits both humoral and cell mediated immune responses with a good neutralization antibody titers. However, these NAb titers were not significantly different from antigen alone, whereas adjuvanted Coronavirus vaccine with formulation 2, induces IFNγ and TNF-α cytokines, indicative of Th1 response. Thus, these, results indicate that the Ad-ISF35 (MemVax) adjuvant drives Th1 mediated immune response, apart from inducing high NAb titers.

We claim:

1. An adjuvanted vaccine formulation against COVID-19 infection, comprising:
a. vaccine antigen, wherein the antigen is an inactivated rabies virus vector expressing S1 domain of SARS-CoV-2 Spike Protein;
b. one or more adjuvants selected from SEPIVAC SWE and MemVax;
c. optionally, one or more stabilizers; and
d. optionally, one or more preservatives.

2. The vaccine formulation as claimed in claim 1, wherein SEPIVAC SWE is an oil in water emulsion adjuvant.

3. The vaccine formulation as claimed in claim 1, wherein MemVax is a replication incompetent adenovirus type 5 (Ad 5), E1 deleted/partial E3 deleted, vector encoding human/mouse chimeric CD40 ligand.

4. The vaccine formulation as claimed in claim 1, wherein the one or more stabilizers are selected from Trehalose, Sucrose, Maltose and Human Serum Albumin (HSA) or combination thereof.

5. The vaccine formulation as claimed in claim 4, wherein the concentration of Trehalose is between 0.5%-5% (w/v); Maltose is between 5% to 10% (w/v); Sucrose is between 0-70% (w/v); and HSA is between 0.5%-5% (w/v).

6. The vaccine formulation as claimed in claim 1, wherein the one or more preservatives are selected from:
0.1% to 0.5% (w/v) 2-phenoxyethanol;
0.003% to 0.01% (w/v) thiomersal;
0.01% to 0.1% (w/v) methyl p-hydroxybenzoate;
0.01 to 0.1% (w/v) propyl p-hydroxybenzoate; and
combinations thereof.

7. The vaccine formulation as claimed in claim 1, wherein the antigen and SEPIVAC SWE and/or MemVax are present in 1:1 ratio (v/v).

8. The vaccine formulation as claimed in claim 1, wherein concentration of the antigen is between 5 to 50 μg.

9. The vaccine formulation as claimed in claim 1, wherein SEPIVAC SWE comprises squalene at a concentration between 1 to 5% (v/v).

10. The vaccine formulation as claimed in claim 9, wherein concentration of squalene in SEPIVAC SWE is between 2.5 to 4.5% (v/v).

11. The vaccine formulation as claimed in claim 1, wherein MemVax comprises of recombinant adenovirus type 5 (Ad 5), vector encoding human/mouse chimeric CD40 ligand between 0.5×10¹⁰ vp/dose to 5×10¹⁰ vp/dose.

12. The vaccine formulation as claimed in claim 11, wherein recombinant adenovirus type 5 (Ad 5), vector encoding human/mouse chimeric CD40 ligand in MemVax is between 0.5×10¹⁰ vp/dose to 3×10¹⁰ vp/dose.

13. The vaccine formulation as claimed in claim 1, wherein the formulation induces robust humoral and cell mediated response against COVID-19 infection compared to antigen alone and provides long term immunity.

14. The vaccine formulation as claimed in claim 1, wherein the formulation elicits high humoral protective response compared to antigen alone in mammals.

15. The vaccine formulation as claimed in claim 1, wherein formulation elicits high Cell meditated or TH1/TH2 immunity compared to antigen alone in mammals.

16. The vaccine formulation as claimed in claim 1, wherein formulation induces TH1 mediated immune response, indicated by robust induction of IFN-γ and TNF-α in mammals.

17. The vaccine formulation as claimed in claim 1, wherein formulation is effective against SARS-CoV-2 variants or variants of Concern (VOCs) such as alpha, Beta, Delta, and omicron.

18. A method of preparing an adjuvanted vaccine formulation against COVID-19 infection, comprising:
a. providing an antigen, wherein the antigen is an inactivated rabies virus vector expressing S1 domain of SARS-CoV-2 Spike Protein; and
b. providing one or more adjuvants selected from SEPIVAC SWE and MemVax;
c. mixing the antigen and adjuvant in 1:1 ratio (v/v);
d. optionally, mixing one or more stabilizers; and
e. optionally, mixing one of more preservatives.

19. The method as claimed in claim 18, wherein SEPIVAC SWE is an oil in water emulsion adjuvant.

20. The method as claimed in claim 18, wherein MemVax is a replication incompetent adenovirus type 5 (Ad 5), E1 deleted/partial E3 deleted, vector encoding human/mouse chimeric CD40 ligand.

21. The method as claimed in claim 18, wherein concentration of the antigen is between 5 to 50 μg.

22. The method as claimed in claim 18, wherein SEPIVAC SWE comprises squalene in a concentration between 1 to 5% (v/v).

23. The method as claimed in claim 22, wherein concentration of squalene in SEPIVAC SWE is between 2.5 to 4.5% (v/v).

24. The method as claimed in claim 18, wherein MemVax comprises of recombinant adenovirus type 5 (Ad 5), vector encoding human/mouse chimeric CD40 ligand between 0.5×10¹⁰ vp/dose to 5×10¹⁰ vp/dose.

25. The method as claimed in claim 24, wherein recombinant adenovirus type 5 (Ad 5), vector encoding human/mouse chimeric CD40 ligand in MemVax is between 0.5×10¹⁰ vp/dose to 3×10¹⁰ vp/dose.

26. The method as claimed in claim 18, wherein the one or more stabilizers are selected from Trehalose, Sucrose, Maltose and Human Serum Albumin (HSA) or combination thereof.

27. The method as claimed in claim 26, wherein the concentration of Trehalose is between 0.5%-5% (w/v); Maltose is between 5% to 10% (w/v); Sucrose is between 0-70% (w/v); and HSA is between 0.5%-5% (w/v).

28. The method as claimed in claim 18, wherein one or more preservatives are selected from:
0.1% to 0.5% (w/v) 2-phenoxyethanol;
0.003% to 0.01% (w/v) thiomersal;
0.01% to 0.1% (w/v) methyl p-hydroxybenzoate;
0.01 to 0.1% (w/v) propyl p-hydroxybenzoate; and
combinations thereof.

29. The use of adjuvants SEPIVAC SWE and/or MemVax in developing adjuvanted COVID-19 vaccine, in which gene of S1 fragment of Spike Protein of SARS-CoV-2 has been inserted between N and P genes of attenuated rabies virus.

30. The use of vaccine formulation as claimed in claim 1, wherein formulation is effective against SARS-CoV-2 variants or variants of Concern (VOCs) such as alpha, Beta, Delta, and omicron.
